(19) United States
(12) Patent Application Publication
(10) Pub. No.: US 2013/0052720 A1
(43) Pub. Date: Feb. 28, 2013

(54) ENZYMATIC PROCESS AND BIOREACTOR USING ELONGATED STRUCTURES FOR CO2 CAPTURE TREATMENT

(71) Applicants: Sylvie Fradette, Pintendre (CA); Anne Belzil, Levis (CA); Melanie Dion, St-Damien-de-Buckland (CA); Romain Parent, Saint Mitre les Remparts (FR)

(72) Inventors: Sylvie Fradette, Pintendre (CA); Anne Belzil, Levis (CA); Melanie Dion, St-Damien-de-Buckland (CA); Romain Parent, Saint Mitre les Remparts (FR)

(73) Assignee: CO2 SOLUTIONS INC., Quebec (CA)

(21) Appl. No.: 13/508,246

(22) PCT Filed: Nov. 4, 2010
(86) PCT No.: PCT/CA10/01787

§ 371 Date: Sep. 24, 2012

Related U.S. Application Data
(60) Provisional application No. 61/272,792, filed on Nov. 4, 2009.

(51) Int. Cl. C12M 1/40; C12S 5/00

(52) U.S. Cl. 435/266; 435/293.1

(57) ABSTRACT

An enzymatic process and bioreactor use elongated structures to enhance CO₂ capture treatments. The enzymatic process and bioreactor treat a fluid by catalyzing reaction (I) with carbonic anhydrase, CO₂+H₂O [symbol] HCO⁻₃+H⁺ (|) by feeding the fluid into a reaction zone wherein a plurality of elongated structures extend through the reaction zone. Each elongated structure supports a flowing liquid layer comprising droplets. Reaction (I) occurs within the flowing liquid layer in the presence of the carbonic anhydrase, to produce a gas stream and a liquid stream which are released. The process and bioreactor can be used in an absorption, desorption or combined treatment context.

### FIELD OF THE INVENTION

The present invention generally relates to the field of CO₂-containing gas treatment. More specifically, the invention relates to a process and a bioreactor using elongated structures to enhance CO₂ capture treatments.

### BACKGROUND TO THE INVENTION

Reduction of Green House Gases (GHG), such as CO₂, is a challenging issue directly involving gas separation and capture processes. A very significant barrier to adoption of carbon capture technology on a large scale is cost of capture. The available technology for conventional CO₂ capture is based primarily on the use of amine solvents within an absorption tower coupled to a desorption (or stripping) tower. This is an energy intensive process that involves heating the solvent to high temperature to strip the CO₂ (and regenerate the solvent) for underground sequestration. The conventional use of amines involves an associated capture cost of approximately US $60 per ton of CO₂ (IPCC), which represents approximately 80% of the total cost of carbon capture and sequestration (CCS), the remaining 20% being attributable to CO₂ compression, pipelining, storage and monitoring. This large cost for the capture portion has, to present, made large scale CCS unviable; based on data from the IPCC, for instance, for a 700 megawatt (MW) pulverized coal power plant that produces 4 million metric tons of CO₂ per year, the capital cost of conventional CO₂ capture equipment on a retrofit basis would be nearly $800 million and the annual operating cost and plant energy penalty would be nearly $240 million. As such, there is a need to reduce the costs of the CO₂ absorption process and develop new and innovative approaches to the problem.

The efficiency of the separation of a particular gas from an effluent gas mixture depends notably on the design of the gas separation reactor. A major limiting factor of gas separation is mass transfer, from the gas phase (effluent gas mixture) into the liquid phase (absorption solution) and vice-versa. Various reactor designs have been proposed to improve this mass transfer.

For example, U.S. Pat. No. 6,582,498 (SASS et al.) discloses the principle of a reactor containing an array of vertical wires down which a liquid solvent flows in drops. SASS et al.'s reactor can be used to absorb CO₂ into a liquid and release CO₂ from an ion loaded liquid. SASS et al. disclose a flow-wire reactor for dissolving gas components such as CO₂ into liquid solvents such as some alkanolamines, and propose the addition of a chemical activator, such as piperazine-based activator, to the liquid solvent to promote the reactions.

Gas separation efficiency may also be improved by the use of biocatalysts, such as enzymes. Enzymes in contact with an absorption solution can catalyze the conversion of absorbed gas compounds into other compounds and thus separate the absorbed compounds from the effluent gas mixture. More particularly, in the case of CO₂ as the absorbed gas compound, carbonic anhydrase can be used to catalyze the hydration reaction of CO₂ as follows:

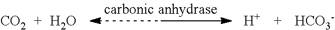

One challenge in the design and operation of enzymatic bioreactors is achieving efficient mass transfer and reaction rates while avoiding reduction of enzyme activity. Enzyme activity can be hampered or even lost due to various factors such as temperature, pressures and destructive forces occurring inside a reactor. Also, different enzymes and modified enzyme variants have different levels of fragility and deactivation to different factors. It is a challenge to strike a balance between enzyme activity and favorable operating conditions for mass transfer and chemical reactions.

There is a need for a technology that raises the efficiency of gas-liquid mass transfer and reduces the cost of CO₂ absorption and/or desorption processes.

### SUMMARY OF THE INVENTION

The present invention responds to the above need by providing an enzymatic process and bioreactor using elongated structures to enhance CO₂ capture treatments.

In one aspect of the present invention, there is provided an enzymatic process for treatment of a fluid by catalyzing reaction (I) with carbonic anhydrase, wherein the reaction (I) is as follows:

CO₂+H₂O [symbol] HCO₃⁻+H⁺ (I)

The enzymatic process comprises:
feeding the fluid into a reaction zone wherein a plurality of elongated structures extend through the reaction zone, each elongated structure supporting a flowing liquid layer comprising droplets therealong;
allowing the reaction (I) to occur within the flowing liquid layer in the presence of the carbonic anhydrase, to produce a gas stream and a liquid stream; and
releasing the gas stream and the liquid stream from the bioreactor.

According to one embodiment of the present invention, the fluid is a CO₂-containing effluent gas and the process comprises feeding an absorption solution into the bioreactor to form the flowing liquid layer along the elongated structures and to contact the CO₂-containing effluent gas so as to dissolve CO₂ from the CO₂-containing effluent gas into the absorption solution. In this embodiment, the reaction (I) is a forward reaction catalyzing the hydration of dissolved CO₂ into bicarbonate ions and hydrogen ions. The gas stream is a CO₂-depleted gas and the liquid stream is an ion-rich solution comprising the bicarbonate ions and hydrogen ions.

According to another embodiment of the present invention, the fluid is an ion-rich solution comprising bicarbonate and hydrogen ions which forms the flowing liquid layer along the elongated structures, and the reaction (I) is a backward reaction catalyzing the desorption of the bicarbonate ions into gaseous CO₂. Thus, the gas stream is a CO₂ stream and the liquid stream is a regenerated solution.

As should be apparent, the process may be an enzymatic absorption and/or desorption process.

In one embodiment, the enzymatic process may be an enzymatic CO₂ absorption process for treatment of a CO₂-containing gas, comprising:
flowing an aqueous absorption solution along a plurality of elongated structures, each elongated structure supporting a flowing liquid layer comprising droplets; and
contacting the flowing liquid layer with the CO₂-containing effluent gas in the presence of carbonic anhydrase, to dissolve the CO₂ into the flowing liquid layer and promote the hydration reaction of the dissolved CO₂ into bicarbonate ions and hydrogen ions, producing a CO₂-depleted gas and an ion-rich solution.

In one aspect of this embodiment, the absorption solution and the CO₂-containing effluent gas flow counter-currently with respect to each other.

In another embodiment, the enzymatic process may also be an enzymatic CO₂ desorption process for treatment of an ion-rich solution comprising bicarbonate ions, comprising:
flowing the ion-rich solution along a plurality of elongated structures, each elongated structure supporting a flowing liquid layer comprising droplets; and
providing carbonic anhydrase in the flowing liquid layer to promote the desorption reaction of the bicarbonate ions to generate CO₂ gas.

In one optional aspect of the process, the flowing liquid layers are managed so as to sheath the elongated structures.

In another optional aspect of the process, the flowing liquid layers are managed so as to be generally discrete with respect to each other.

In another optional aspect of the process, the flowing liquid layers are parallel with respect to each other.

In another optional aspect of the process, the flowing liquid layers flow in a generally straight direction.

In another optional aspect of the process, the flowing liquid layers flow downward.

In another optional aspect of the process, the carbonic anhydrase is provided free in the flowing liquid layers.

In another optional aspect of the process, the carbonic anhydrase is provided on or in particles that are in the flowing liquid layers.

In another optional aspect of the process, the fluid further comprises at least one chemical compound selected from alkanolamines and amino acids.

In another embodiment of the present invention, there is provided an enzymatic bioreactor for treatment of a fluid with carbonic anhydrase, the bioreactor comprising:
a reaction chamber having side walls and two opposed ends defining a reaction zone therewithin;
a fluid inlet in fluid communication with the reaction chamber for feeding the fluid into the reaction zone;
a plurality of elongated structures extending between the two opposed ends through the reaction zone, each elongated structure supporting a flowing liquid layer comprising droplets therealong wherein a reaction (I) occurs within the flowing liquid layer in the presence of the carbonic anhydrase and catalyzed thereby:

CO₂+H₂O [symbol] HCO₃⁻+H⁺ (I)
thereby producing a gas stream and a liquid stream;
a liquid outlet in fluid communication with the reaction chamber for releasing the liquid stream; and
a gas outlet in fluid communication with the reaction chamber for releasing the gas stream.

In one optional aspect of the bioreactor, the elongated structures are cylindrical.

In another optional aspect of the bioreactor, the elongated structures are wires.

In another optional aspect of the bioreactor, the elongated structures are spaced apart and parallel with respect to each other.

In another optional aspect of the bioreactor, the elongated structures are linear.

In another optional aspect of the bioreactor, the elongated structures have an upright orientation and the flowing liquid layers flow down the elongated structures.

In another optional aspect of the bioreactor, the elongated structures are evenly spaced away from each other and from the side walls and substantially fill the reaction zone.

In another optional aspect of the bioreactor, the elongated structures each comprise outer surfaces which support the flowing liquid layer such that the flowing liquid layer takes the form of an annular channel comprising annular droplets, sheathing the outer surfaces.

In another optional aspect of the bioreactor, the elongated structures each have opposed extremities that are respectively mounted to the opposed ends of the reaction chamber.

In another optional aspect of the bioreactor, the carbonic anhydrase is provided free in the flowing liquid layers.

In another optional aspect of the bioreactor, the carbonic anhydrase is provided on or in particles that are in the flowing liquid layers.

In another optional aspect of the bioreactor, the fluid further comprises at least one chemical compound selected from alkanolamines and amino acids.

In another optional aspect of the bioreactor, the enzymatic bioreactor comprises a gas inlet receiving a CO₂-containing effluent gas and the liquid inlet receives an absorption solution, the gas inlet and the liquid inlet being provided respectively at a bottom and a top of the reaction chamber, such that the absorption solution and the CO₂-containing effluent gas flow counter-currently with respect to each other.

The enzymatic process and bioreactor use the elongated structures to support the flowing liquid layer so as to promote efficient mass transfer and enzymatically catalyzed reactions while allowing a flow regime favourably accommodating the carbonic anhydrase enzyme.

### BRIEF DESCRIPTION OF THE DRAWING

Embodiments of the bioreactor and the enzymatic process, according to the present invention, are represented in the following figures.

FIG. 1 is a vertical cross-section schematic view of an absorption bioreactor according to an embodiment of the present invention.

FIG. 2 is a vertical cross-section schematic view of a desorption bioreactor according to another embodiment of the present invention.

FIG. 3 is a process flow diagram of a process according to an embodiment of the present invention.

FIG. 4 is a close-up partial cross-section schematic view of an elongated structure and flowing liquid layer comprising droplets according to an embodiment of the present invention.

FIG. 5 is a vertical cross-section schematic view of an absorption bioreactor according to an embodiment of the present invention.

FIG. 6 is a vertical cross-section schematic view of a desorption bioreactor according to another embodiment of the present invention.

FIG. 7 is a vertical cross-section schematic view of an absorption bioreactor according to yet another embodiment of the present invention.

FIG. 8 is a vertical cross-section schematic view of a desorption bioreactor according to yet another embodiment of the present invention.

While the invention will be described in conjunction with example embodiments, it will be understood that it is not intended to limit the scope of the invention to these embodiments. On the contrary, it is intended to cover all alternatives, modifications and equivalents as may be included as defined by the appended claims.

### DETAILED DESCRIPTION

The present invention provides enzymatic processes and bioreactors for CO₂ capture treatments, which use elongated structures to support flowing liquid layers comprising droplets to provide a flow regime for enhanced enzyme catalyzed reactions, e.g. reaction (I) as follows:

CO₂+H₂O [symbol] HCO₃⁻+H⁺ (I)

In one embodiment of the present invention shown on FIG. 1, the bioreactor is an absorption reactor (2). The absorption reactor (2) has a reaction chamber (4) which has a reaction zone (6) defined therein. There is also a plurality of elongated structures (8) within the reaction zone (6). The absorption reactor (2) also has a gas inlet and a liquid inlet. The absorption reactor (2) is fed with an absorption solution (10) and a CO₂-containing gas (12). The gas (12) contacts the absorption solution (10) which flows down the elongated structures (8). The elongated structures (8) may be arranged vertically as shown in FIG. 1 or slightly inclined, preferably with their extremities mounted to the opposed ends of the reaction chamber (4). Preferably, the elongated structures (8) are spaced away from each other as shown in FIG. 1. It should be noted that the elongated structures may have an inter-spacing designed to favor certain flow characteristics. The reaction zone may have an amount of elongated structures (8) depending on the size of the reaction zone and the spacing between the elongated structures (8). The liquid absorption solution (10) entering the reaction chamber (4) through the liquid inlet preferably situated at the bottom of the reaction chamber (4), is an aqueous solution capable of absorbing CO₂ (also referred further below as an ion-lean solution). The gas stream (12) enters the bioreactor through an inlet preferably situated at the bottom of the reaction chamber (4). This gas stream (12) is a CO₂-containing gas mixture which may come from any number of sources such as industrial or power plant sources. The CO₂-containing gas mixture (12) and the absorption solution (10) may be distributed within the reaction chamber (4) through perforated distribution plates (14a and 14b) respectively placed at the bottom and the top of the reaction chamber (4). The absorption solution (10) reacts with the CO₂-rich gas mixture (12) within the reaction chamber (4) and more particularly, within the reaction zone (6) situated in between the two perforated distribution plates (14a and 14b). The perforations (quantity, size, shape, distribution, etc.) enable the control of the fluid flow to maintain adequate or desired hydrodynamics.

Referring briefly to FIG. 4, enzymes (16) are provided so as to catalyze the desired reactions. In the case of the absorption bioreactor (2), carbonic anhydrase catalyzes the hydration reaction of CO₂ into bicarbonate and hydrogen ions. The enzymes are preferably provided in the absorption solution and flow therewith or may be already present within the bioreactor to catalyze the reaction. Each elongated structure (8) supports a flowing liquid layer (18) comprising droplets (20). The elongated structures (8) may be spaced apart from each other and configured such that the droplets (20) of one flowing liquid layer (18) tend not to contact the droplets of adjacent elongated structures. The elongated structures (8) may also be sized to promote distinct flowing liquid layers and surface area in contact with the gas phase. For example, the cross-sectional diameter of the elongated structures may be sized to minimize the thickness of the flowing liquid layer and the size of the droplets. The CO₂ is absorbed into the flowing liquid layer (18) of absorption solution (10) flowing along the elongated structures (8) and the CO₂-containing gas is thus purified into a CO₂-depleted gas (22) released from the absorption bioreactor (2) through a gas outlet preferably situated at the top of the reaction chamber (4). The absorbed CO₂ is converted into bicarbonate and hydrogen ions transforming the absorption solution (10) into an ion-rich solution (24) which is released from the absorption bioreactor (2) through a liquid outlet situated at the bottom of the reaction chamber (4). The ion-rich solution (24) containing the product of the enzymatic reaction is preferably directed towards a treatment unit for use, valorization or extraction of this product. For example, the exiting ion-rich solution (24) can be subjected to a reaction of its bicarbonate ions with a cation such as calcium or magnesium to generate a precipitate, or can undergo desorption, in order to regenerate fresh absorption solution and enable its recirculation.

In some embodiments, the present invention provides a gas-liquid bioreactor internally equipped with a plurality of elongated structures in which enzymes are provided, directly via an absorption solution or immobilized within the reactor. An objective of such a reactor is to enable the enzymatic process of separation of carbon dioxide (CO₂) contained in an effluent gas mixture. The bioreactor promotes good separation performance and high energy efficiency due to various characteristics.

The architecture of the bioreactor with a plurality of elongated structures enables hydrodynamics that are favorable to CO₂ mass transfer. This configuration of the bioreactor also enables an improvement in terms of energy loss (pressure losses, etc.) compared to packed columns. The conversion of CO₂ into bicarbonate and hydrogen ions takes place in the presence of enzymes, preferably carbonic anhydrase, thereby producing a CO₂-depleted gas and an ion-rich solution.

The specific hydrodynamic flow, proper to the presence of elongated structures, creates instability by the formation of drops of absorption solution that flow along the elongated structures. The surface of the drops offers a large CO₂ mass transfer interface which is continuously renewed with fresh absorption solution while it flows along the elongated structures. Preferably, the droplets are small to provide a better exchange interface and improved CO₂ mass transfer.

In addition, the presence of the enzyme within the enzymatic bioreactor enables a reaction of conversion of CO₂ into ions that is both fast and selective. This acceleration of the reaction also contributes to the improvement of the CO₂ mass transfer. Indeed, an improvement brought by the enzyme includes the rapid transformation of the CO₂, which accordingly decreases its concentration in the drops of absorption solution formed along the elongated structures. The exposed liquid surfaces are renewed with new small drops of fresh absorption solution, taking the place of other drops which have already reacted with the incoming CO₂; the CO₂ concentration gradient is thus maintained at a high level.

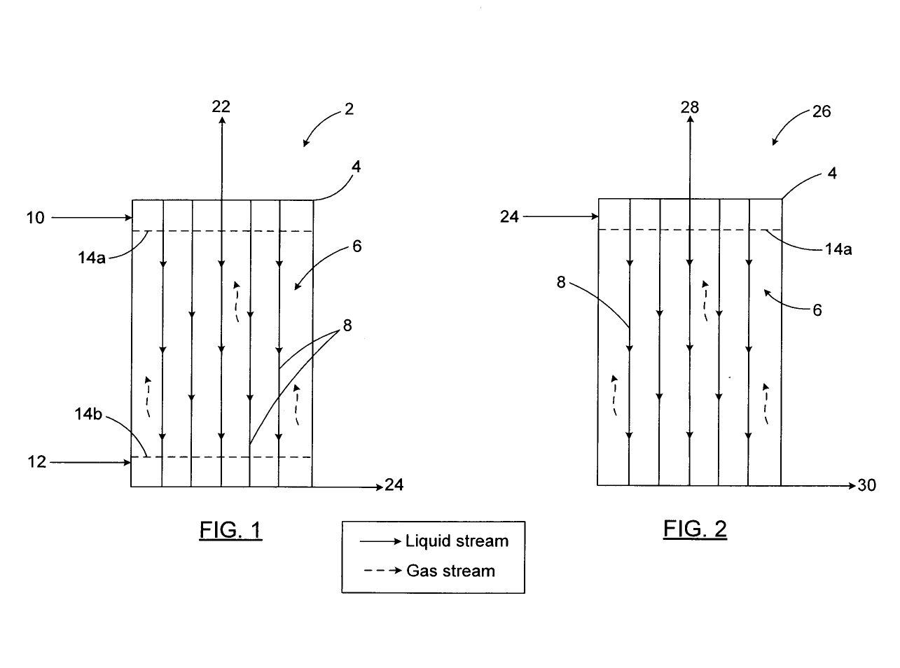

According to another embodiment of the present invention shown in FIG. 2, the bioreactor may be a desorption reactor (26) used to recover gaseous CO₂ from an ion-loaded solution, which may be the ion-rich solution (24) from the absorption reactor (2). The ion-rich solution (24) enters the bioreactor through a liquid inlet preferably situated at the top of the reaction chamber (4) and is distributed through a perforated distribution plate (14a). The ion-rich solution (24) is preferably heated to favor the desorption process. The enzymes, such as carbonic anhydrase, may be present within the ion-rich solution (24) and promote the conversion of the bicarbonate ions into regenerated CO₂ gas (28), producing an ion-lean solution (30) which may be recycled as absorption solution (10). The regenerated CO₂ gas (28) can be thus separated for sequestration, storage or various uses.

Referring to FIG. 4, each elongated structure (8) supports the flowing liquid layer (18) of absorption solution or ion-rich solution which is in direct contact with the surrounding gas. This allows absorption of the CO₂ at the surface of the flowing liquid layer (18) for an absorption process and allows desorption of CO₂ out of the flowing liquid layer (18) for a desorption process. The enzymes (16), such as carbonic anhydrase, may be flowing freely within the flowing liquid layer (18) as illustrated and can catalyze the desired reactions. When the enzymes are provided within the flowing liquid layer (18), either free or supported by particles, they flow and are distributed throughout the flowing liquid layer and its droplets to facilitate catalysis within the flowing liquid layer. Alternatively, the enzymes may be immobilized to the elongated structures, in which case the gaseous CO₂ is quickly dissolved into the drops to react, transported to the surface of the elongated structures for hydrolysis, and the reactants are quickly transported away from the elongated structures with the flowing of the drops, thus avoiding accumulation of reactant ions at the structure surfaces.

The enzyme may be immobilized on or sequestered in the material of the elongated structures. An enzymatic layer (continuous or not) of particles and/or any physical forms (nanotubes, for example, or any other forms) may be fixed, deposited or glued to the elongated structures by chemical, electrostatic or physical means. The enzyme may be provided free in the liquid solution forming the flowing liquid layer; immobilized on the surface of supports that are mixed in the absorption solution and are flowable therewith; entrapped or immobilized by or in porous supports that are mixed in the absorption solution and are flowable therewith; as cross-linked enzyme aggregates (CLEA) or crystals (CLEC) flowing therewith; or a combination thereof.

The enzyme may be supported by particles, such as micro-particles or nanoparticles, which are carried with the absorption solution. For the absorption unit the particles may be sized in accordance with the reactive film at the surface of the droplets which is approximately 10 microns, and thus may be sized to be smaller than 10 microns. The particles are also sized so as to be smaller than the minimal thickness of the flowing liquid layer. Enzymes and enzymatic particles provided so as to flow within the flowing liquid layer are subjected to the flow regime of the flowing liquid layer, rather than the flow regime that would be present in a packed column reactor. The flow regime enabled by the elongated structures may allow various support materials, immobilization materials and enzyme aggregate or crystal systems, to experience reduced deterioration and the corresponding impairment of enzyme stabilization and functionality due to such deterioration, as the case may be.

In the case of CO₂, carbonic anhydrase is used in most cases since this enzyme catalyses the hydration reaction of CO₂. Other types of enzymes can also be envisioned and provided for other types of gas-liquid reactors that are similar to the CO₂ capture processes described herein. Different enzymes can be provided alone or combined together in other embodiments of the bioreactor.

The elongated structures (8) may be composed of wettable material (cotton or metal, strands of silicone or polymer fibres, for example) or may be covered by a wettable film. The length, the diameter and the number of elongated structures are variable and may be designed or adjusted according to the required specifications of the separation process. The same can be said for the arrangement and spacing of the elongated structures in the reaction chamber. The elongated structures can be wires with mono-filaments or multi-filaments, with or without torsion, cylindrical and linear or of irregular shape.

The flow regime can also be influenced by providing perturbations, to destabilize or otherwise enhance the flow and mass transfer. For example, physical obstacles may be placed along the elongated structures. The size and form of the obstacles can vary. Other perturbations can be created by mechanical systems enabling, for example, a torsion of the elongated structure or a vibration of the elongated structure in its vertical or orthogonal axis. These structural or mechanical perturbations can enable the formation of more desirable flowing liquid layer along the elongated structures to improve CO₂ mass transfer.

The absorption solution (10) that is used to feed the absorption bioreactor (2) may be of any kind as long as it presents the capacity to absorb the CO₂ to be separated and enables the activity of the enzyme. Preferably, it is an aqueous solution containing one absorption compound or a mix of absorption components, for example a mix of amines. Amines are often used in effluent treatment processes due to their absorptive and reactive properties as well as their miscibility with water. Examples of common amine solvent absorbents are monoethanolamine (MEA), 2-amino-2-hydroxymethyl-1,3-propanediol (TRIS), among others. The absorption solution may comprise a carbonate compound, an amino-acid compound or a combination thereof. The carbonate compound may comprise potassium carbonate, sodium carbonate or ammonium carbonate while the amino-acid compound may comprise at least one primary, secondary and/or tertiary amino acid, derivative thereof, salt thereof and/or mixture thereof. More particularly, the amino-acid may comprise at least one of the following: glycine, proline, arginine, histidine, lysine, aspartic acid, glutamic acid, methionine, serine, threonine, glutamine, cysteine, asparagine, valine, leucine, isoleucine, alanine, valine, tyrosine, tryptophan, phenylalanine; taurine, N, cyclohexyl 1,3-propanediamine, N-secondary butyl glycine, N-methyl N-secondary butyl glycine, diethylglycine, dimethylglycine, sarcosine, methyl taurine, methyl-α-aminopropionic acid, N-(3-ethoxy)taurine, N-(β-aminoethyl)taurine, N-methyl alanine, 6-aminohexanoic acid; or alkali salts thereof; or a combination thereof. The absorption solution may also comprise an absorption compound such as piperidine, piperazine and derivatives thereof which are substituted by at least one alkanol group, alkanolamines, monoethanolamine (MEA), 2-amino-2-methyl-1-propanol (AMP), 2-(2-aminoethylamino)ethanol (AEE), 2-amino-2-hydroxymethyl-1,3-propanediol (Tris).

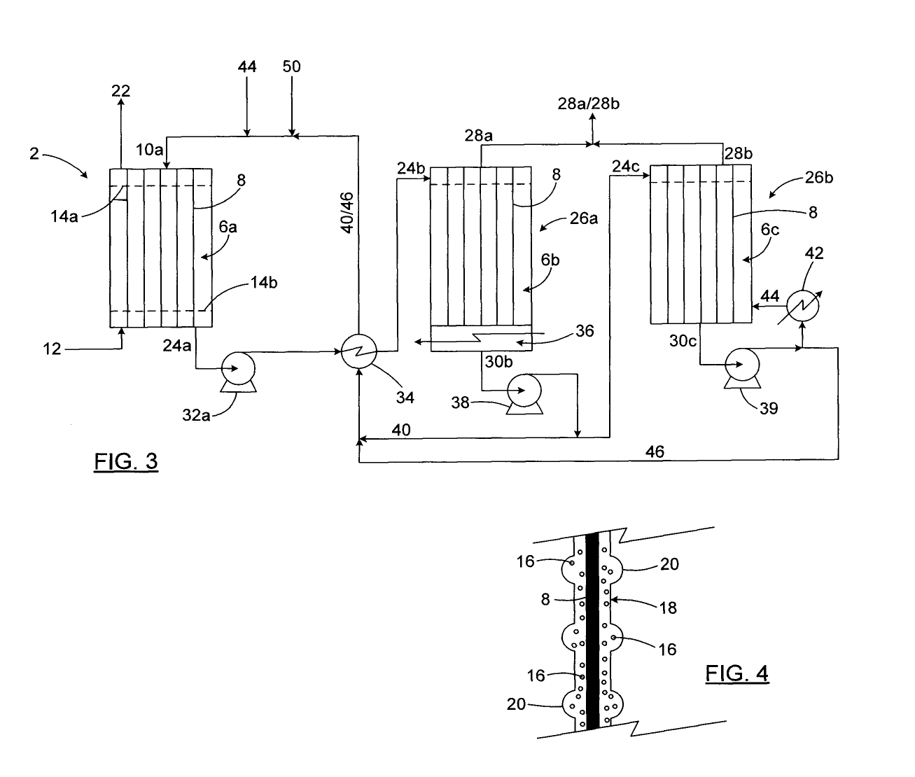

FIG. 3 shows another embodiment including both absorption and desorption units. In this process, multiple desorption reactors (26a, 26b) may be used in series with an absorption reactor (2) in order to capture CO₂ and recycle various streams back into the process. The CO₂-containing gas mixture (12) enters the absorption reactor (2) and contacts an absorption solution (10a). The purified gas (22) depleted of CO₂ exits the absorption reactor (2). In the presence of enzymes, the absorbed CO₂ is converted into bicarbonate and hydrogen ions, thereby producing an ion-rich solution (24a). Two types of desorption reactors (26a and 26b) may follow. The ion-rich solution (24a) is pumped by a pump (32a) to the first desorption reactor (26a) and is heated through a heat exchanger (34). The desorption reactor (26a) receives the heated ion-rich solution (24b) which flows down along the elongated structures (8) and may be reboiled by a reboiler (36) directly present within the desorption reactor (26a). This additional heating promotes an efficient desorption of the CO₂. The ion-depleted solution (30b) is pumped by a pump (38) and may be split into two liquid streams (40 and 14c). A gaseous CO₂ stream (28a) is released through an outlet situated at the top of the desorption reactor (26a). The second desorption reactor (26b) receives a solution still containing some ions (14c) that may undergo desorption and produce further desorbed CO₂ gas (28b). The solution (14c) flows along the elongated structures (8) and becomes a further ion-lean solution (30c) while gaseous CO₂ is desorbed. This second desorption reactor (26b) includes a reboiler (42), which takes a fraction of the ion-lean absorption solution (30c) fed by a pump (39) and recycles it into the second desorption reactor (26b) after having heated it to produce a heated solution (44) comprising steam. This steam will create a driving force such that CO₂ will be further released from the entering solution (14c). The two fractions of ion-lean solution (40 and 46) exiting the two desorption reactors (26a and 26b) are preferably recycled to the absorption reactor (2). Their heat may be transmitted to the ion-rich solution (24a) through the heat exchanger (34) to save energy. Fresh water (48) can be added to the incoming absorption solution (10a) in order to compensate for the natural evaporation losses. Fresh enzymes (50) may also be added, which may be in an aqueous form or in dry form.

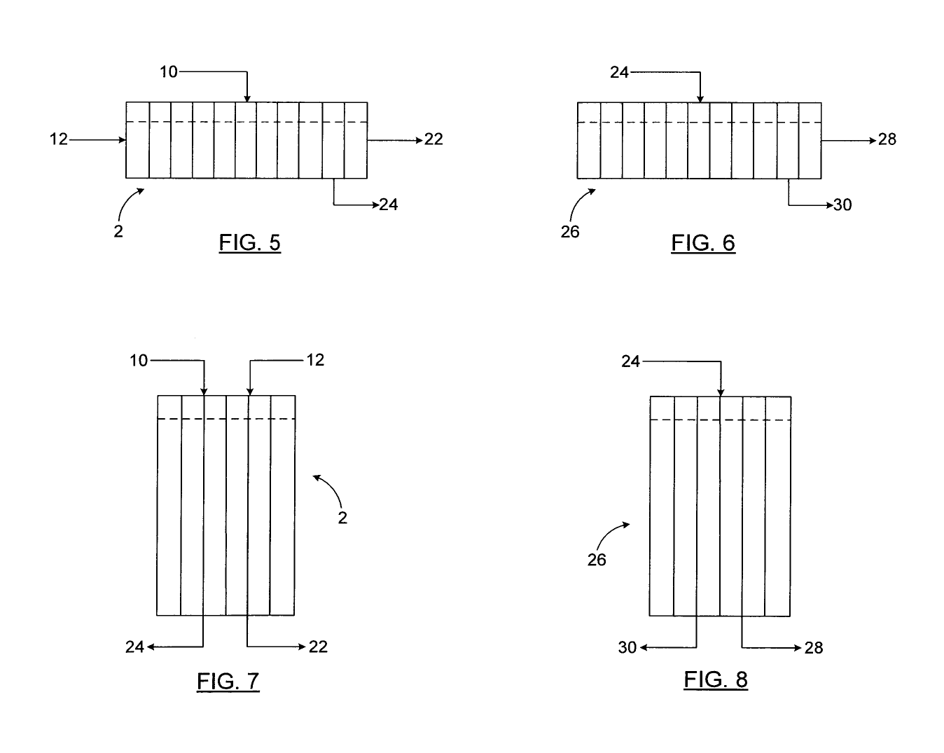

Referring now to FIGS. 5, 6, 7 and 8, various configurations of absorption reactors (2) and desorption reactors (26) are considered. The embodiments of the present invention previously shown in FIGS. 1, 2 and 3, correspond to a situation where the gas stream flows counter-currently with the liquid stream. In FIGS. 5 and 6, the liquid streams (10, 24) flow cross-currently to the respective gas stream (12, 22, 28). In FIGS. 7 and 8, the liquid streams (10, 24) flows co-currently with the respective gas stream (12, 22, 28). Furthermore, the flow rates and retention times of the gas stream and liquid stream may be determined so as to optimize the purification process dependant on operating conditions, conduit dimensions, and other features of the units that make up the system.

The present invention includes an enzymatic process to treat a fluid, such as a CO₂-containing effluent gas or an ion-rich solution using enzyme catalysis and elongated structures supporting flowing liquid layers where the reactions take place. The process is catalyzed by an enzyme such as carbonic anhydrase. The present invention also provides the combination of enzymes with a reactor internally equipped with elongated structures, forming an enzymatic bioreactor with hydrodynamics favorable to CO₂ mass transfer and enzyme activity. Other enzymes may be used to catalyze other reactions to separate a component from one phase to another.

The following references are incorporated herein by reference and it should be understood that various aspects described therein may be combined with various aspects of the described herein: PCT/CA2010/001212, PCT/CA2010/001213, PCT/CA2010/001214, U.S. Pat. No. 6,908,507, U.S. Pat. No. 7,176,017, U.S. Pat. No. 6,524,843, U.S. Pat. No. 6,475,382, U.S. Pat. No. 6,946,288, U.S. Pat. No. 7,596,952, U.S. Pat. No. 7,740,689, U.S. Pat. No. 7,514,056, U.S. Pat. No. 7,521,217, U.S. 61/272,792 which are all currently held by the Applicant. The reactors and processes described in the preceding references may be used in connection with the processes described herein. For example, there may be an absorption-desorption CO₂ capture process in which a reactor of the present invention is used as the absorption bioreactor and a packed tower, or spay tower or other type of reactor is used as the desorption bioreactor. In addition, an absorption or desorption bioreactor may be designed so as to have multiple compartments or sections, elongated structures being provided in one section and the other section having a different design such as a packed section, spray section, fluidized bed section, and so on, and the multiple sections may be mounted and interfaced together in an appropriate manner. All other patents, applications and publications mentioned above are hereby also incorporated herein by reference.

Although preferred embodiments of the present invention have been described herein in detail and illustrated in the accompanying drawing, it is to be understood that the invention is not limited to these specific embodiments and that various changes and modifications may be effected thereto without departing from the scope or spirit of the present invention.

1. An enzymatic process for treatment of a fluid by catalyzing reaction (I) with carbonic anhydrase, wherein the reaction (I) is as follows:
CO₂+H₂O [symbol] HCO₃⁻+H⁺ (I)
the process comprising:
feeding the fluid into a reaction zone comprising a plurality of elongated structures extending between the two opposed ends through the reaction zone, each elongated structure supporting a flowing liquid layer comprising droplets therealong;
allowing the reaction (I) to occur within the flowing liquid layer in the presence of the carbonic anhydrase, to produce a gas stream and a liquid stream; and
releasing the gas stream and the liquid stream from the reaction zone.

2. The process of claim 1, wherein:
the fluid is a CO₂-containing effluent gas;
the process comprises feeding an absorption solution into the bioreactor to form the flowing liquid layer along the elongated structures and to contact the CO₂-containing effluent gas so as to dissolve CO₂ from the CO₂-containing effluent gas into the absorption solution;
the reaction (I) is a forward reaction catalyzing the hydration of dissolved CO₂ into bicarbonate ions and hydrogen ions; and
the gas stream is a CO₂-depleted gas and the liquid stream is an ion-rich solution comprising the bicarbonate ions and hydrogen ions.

3. The process of claim 2, wherein the absorption solution and the CO₂-containing effluent gas flow counter-currently with respect to each other.

4. The process of claim 1, wherein:
the fluid is an ion-rich solution comprising bicarbonate and hydrogen ions;
the ion-rich solution forms the flowing liquid layer along the elongated structures; and
the reaction (I) is a backward reaction catalyzing the desorption of the bicarbonate ions into gaseous CO₂;
the gas stream being a CO₂ stream and the liquid stream being a regenerated solution.

5. The process of claim 1, comprising managing the flowing liquid layers so as to sheath the elongated structures.

6. The process of claim 1, comprising managing the flowing liquid layers so as to be generally discrete with respect to each other.

7. The process of claim 1, wherein the flowing liquid layers are parallel with respect to each other.

8. The process of claim 1, wherein the flowing liquid layers flow in a generally straight direction.

9. The process of claim 1, wherein the flowing liquid layers flow downward.

10. The process of claim 1, wherein the carbonic anhydrase is provided free in the flowing liquid layers.

11. The process of claim 1, wherein the carbonic anhydrase is provided on or in particles that are in the flowing liquid layers.

12. The process of claim 1, wherein the fluid further comprises at least one chemical compound selected from alkanolamines and amino acids.

13. An enzymatic bioreactor for treatment of a fluid with carbonic anhydrase, the bioreactor comprising:
a reaction chamber having side walls and two opposed ends defining a reaction zone therewithin;
a fluid inlet in fluid communication with the reaction chamber for feeding the fluid into the reaction zone;
a plurality of elongated structures extending between the two opposed ends through the reaction zone, each elongated structure supporting a flowing liquid layer comprising droplets therealong wherein a reaction (I) occurs within the flowing liquid layer in the presence of the carbonic anhydrase and catalyzed thereby:
CO₂+H₂O [symbol] HCO₃⁻+H⁺ (I)
thereby producing a gas stream and a liquid stream;
a liquid outlet in fluid communication with the reaction chamber for releasing the liquid stream; and
a gas outlet in fluid communication with the reaction chamber for releasing the gas stream.

14. The enzymatic bioreactor of claim 13, wherein the elongated structures are cylindrical.

15. The enzymatic bioreactor of claim 13, wherein the elongated structures are wires.

16. The enzymatic bioreactor of claim 13, wherein the elongated structures are spaced apart and parallel with respect to each other.

17. The enzymatic bioreactor of claim 13, wherein the elongated structures are linear.

18. The enzymatic bioreactor of claim 13, wherein the elongated structures have an upright orientation and the flowing liquid layers flow down the elongated structures.

19. The enzymatic bioreactor of claim 13, wherein the elongated structures are evenly spaced away from each other and from the side walls and substantially fill the reaction zone.

20. The enzymatic bioreactor of claim 13, wherein the elongated structures each comprise outer surfaces which support the flowing liquid layer such that the flowing liquid layer takes the form of an annular channel comprising annular droplets, sheathing the outer surfaces.

21. The enzymatic bioreactor of claim 13, wherein the elongated structures each have opposed extremities that are respectively mounted to the opposed ends of the reaction chamber.

22. The enzymatic bioreactor of claim 13, wherein the carbonic anhydrase is provided free in the flowing liquid layers.

23. The enzymatic bioreactor of claim 13, wherein the carbonic anhydrase is provided on or in particles that are in the flowing liquid layers.

24. The enzymatic bioreactor of claim 13, wherein the fluid further comprises at least one chemical compound selected from alkanolamines and amino acids.

25. The enzymatic bioreactor of claim 13, comprising a gas inlet receiving a CO₂-containing effluent gas and the liquid inlet receives an absorption solution, the gas inlet and the liquid inlet being provided respectively at a bottom and a top of the reaction chamber, such that the absorption solution and the CO₂-containing effluent gas flow counter-currently with respect to each other.
